(19) United States
(12) Patent Application Publication
(10) Pub. No.: US 2007/0129674 A1
(43) Pub. Date: Jun. 7, 2007

(54) Latching blocking mechanisms and safety medical needle assemblies

(76) Inventor: Barry Peter Liversidge, Colchester (GB)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 10/575,037

(22) PCT Filed: Oct. 8, 2004
(86) PCT No.: PCT/GB04/04252

§ 371 Date: Apr. 6, 2006

(30) Foreign Application Priority Data
Oct. 9, 2003 (GB) 03 23653.6
Mar. 23, 2004 (GB) 04 06458.0

(51) Int. Cl. A61M 5/00; A61M 5/32

(52) U.S. Cl. 604/110; 604/198

(57) ABSTRACT

A latching blocking mechanism includes a static component (10,50), a movable component (14,52) slidable relative to the static component and a control member (21,58) arranged to slide with the movable component (14,52) but displaceable transversely or rotationally with respect to the movable component. After the control member (21,58) has moved from a first position through a pre-determined distance towards a second position, a spring (F,60) becomes active to urge the control member to its second position. Sliding movement of the movable component (14,52) displaces the control member (21,58) in the sliding direction to interengage respective camming parts (12,25) on the static component (10,50) and control member (21,58) thereby to displace the control member transversely through said pre-determined distance. Return of the movable component (14,52) to its initial position allows the control member (21,58) to move to its second position under the action of the spring (F,60) thereafter to block movement of the movable component away from its initial position.

This invention relates to latching blocking mechanisms, arranged to block movement of a movable component following the performance of a cycle of movement of that component. The invention further relates to safety medical needle assemblies (whether or not actually including a medical needle) and which incorporate a latching blocking mechanism of this invention. The invention also relates to syringe apparatus comprising a syringe in conjunction with safety medical needle assembly of this invention.

In its preferred aspects, the invention concerns apparatus for using a medical needle having a mount end and a sharp tip, and intended for penetration of a human or animal body, or for other medical uses such as the penetration of a pierceable membrane of an intravenous medication system. For convenience, in the following all such medical uses will be described simply as the penetration of a body, even though specific embodiments may be intended for other medical uses. In addition, though the latching blocking mechanisms may be used in other industries besides the intended medical use, the mechanisms will primarily be described with reference to that medical use. Despite this, in its broader aspects the invention is not to be regarded as limited solely to medical uses.

Liquids of various kinds may be administered to a human or animal body by means of a hollow needle in conjunction with a source of the required liquid. For example, such a needle may be used in conjunction with a syringe holding a liquid drug, the needle being used to penetrate the body at the site at which the drug is to be received. Equally, body fluids may be withdrawn by using a hollow needle which is used to penetrate the body until the tip is located at the site from which fluid is to be withdrawn.

A recognised hazard for clinicians and other persons using medical needles for the above described purposes is the risk of a so-called needle-stick injury—that is to say the accidental penetration of the clinician's skin by the needle. Prior to the use of the needle to supply a liquid to or to withdraw liquid from a body, this rarely presents much of a problem, though once the needle has been used on a body, there is a very much higher risk of a serious consequence for the clinician, or others associated with the disposal of a used needle. During use of the needle to penetrate the body tissues of a patient, the needle is likely to become contaminated with various organisms and should a needle-stick injury occur, these could infect the clinician or other person suffering the needle-stick injury.

There have been numerous proposals for protecting the sharp tip of a used needle, in order to reduce the risk of a needle-stick injury following use of the needle. Some proposals have actually increased the likelihood of such an injury by virtue of the action which must be performed to protect the tip, even if the risk thereafter is lessened. Despite all of the proposals which have previously been made, very few have achieved commercial success, nor has there been wide acceptance by the medical industry. Many proposals are somewhat complex and involve a significantly greater manufacturing cost, and so are unacceptable on economic grounds. Others are much more difficult to use as compared to an unprotected needle, and so are rejected by clinicians. Yet further proposals do not allow compliance with best practice protocols.

There is a significant demand for a protective device for use with a needle, and which allows a clinician or perhaps a patient (in the case of self-administration) to use the needle in much the same way as is done with an unprotected needle, but which can be manufactured economically and which provides a high degree of protection against needle-stick injury. In this connection, it is highly preferred that the device operates fully automatically, without intervention by the user, to give a degree of protection to the needle tip before use, and after use wholly to prevent access to the needle tip other than by a determined attempt to override the protection. In this way, protection may be afforded not just to the user, but also to all others who could come into a risky situation with used needles, such as waste disposal operators, cleaners, and so on.

A device which protects a needle tip without an operator having to perform any extra step on withdrawing the needle from a body is usually referred to as a passive protection device. This may be contrasted with an active protection device, where an operator is required to perform an extra step in order to protect a needle, following the withdrawal of the needle from a body. The requirement to perform an extra step leaves the needle unprotected for a longer period than with a passive protection device and further the performance of that extra step often exposes the user to a potentially hazardous situation, where needle-stick injuries can occur.

Consequent upon research and development, the present invention has evolved, to provide a latching blocking mechanism able to prevent the movement of a component (such as a protective sleeve for a needle) following the cycling of that component through a range of movement (such as to expose the needle, and then to enclose it again). That blocking mechanism may be used in various industrial situations, but a particularly preferred use is in providing a medical needle protection device having enhanced characteristics, but employing the same underlying passive protection concept.

According to a first aspect of this invention, there is provided a latching blocking mechanism, comprising:

a static component including an elongate guide;

a static abutment surface;

a movable component slidable along the guide between initial and shifted positions;

a control member arranged for movement with the movable component in the sliding direction thereof but displaceable with respect thereto in a direction transverse to the sliding direction of the movable component, the control member initially being disposed at a first position relative to the movable component;

control means arranged to urge the control member transversely towards a second position from the first position, which control means becomes active only after the control member has moved by a predetermined distance from its first position towards its second position; and

a static camming part co-operable with a moving camming part on the control member and arranged to move the control member through said predetermined distance from its first position on movement of the movable component from its initial position towards its shifted position;

whereby following the movement of the control member through said predetermined distance and the return of the movable component towards its initial position, the control member is driven by the control means to a blocking position between the static abutment surface and the movable component thereafter to block further movement of the movable component towards its shifted position.

Throughout this specification, reference is made to various parts as being “static” and “movable” or “moving”. It will be appreciated that these terms are relative and a part which is “movable” moves with respect to a “static” part—but equally, a “movable” part could remain stationary with respect to the ground, while a “static” part moves relative thereto.

The mechanism of this invention allows the movable component to be cycled through a pre-set distance by sliding along the guide and back again, whereafter the control member is driven to a blocking position where the movable component is blocked from moving towards its shifted position for a second time. The control member is latched in that blocking position so that it cannot readily be moved back to its first position, so effectively preventing the movable component from being moved once more, from its initial position. Preferred embodiments of this invention permit the movable component to be cycled indefinitely through less than said pre-set distance without the blocking effect taking place, and only when the component is cycled through the full pre-set distance is the blocking action triggered.

The latching blocking mechanism may be arranged linearly, or in a circular form. In the latter case, there may be two, three or even more similar mechanisms arranged in a circular disposition and all working simultaneously together, to achieve an effective and secure blocking action for a sleeve-like movable component. A particularly preferred form of the mechanism when arranged in a circular disposition may be used to provide a safety medical needle assembly, wherein a sleeve surrounds a medical needle but which sleeve may be slid with respect to the needle to a shifted position so as to expose the needle and may then be slid back to a protecting position—but when in that protecting position, the sleeve is blocked there and cannot be slid to the shifted position for a second time, to expose the needle again.

Thus, according to a second aspect, this invention provides a safety medical needle assembly, comprising:

a tubular component including an internal elongate guide;

an abutment surface formed on the tubular component;

a movable component disposed within the tubular component and arranged for axial sliding movement with respect thereto between initial and shifted positions, the movable component being adapted to receive the hub of a medical needle so that the needle projects within but is protected by the movable component when in its initial position;

a control member arranged for axial movement with the movable component but rotatable with respect to the movable component, the control member initially being disposed at a first position relative to the movable component;

control means arranged to urge the control member towards a second position angularly displaced from the first position, which control means becomes active only after the control member has turned through a predetermined angle towards the second position; and

a camming part on the tubular component co-operable with a moving camming part on the control member and arranged to turn the control member through said predetermined angle from its first position on axial movement of the movable component towards its shifted position to cause the needle to project from the movable component;

whereby the movement of the movable component towards its shifted position to cause the needle to project therefrom also causes the control member to turn through said predetermined angle whereafter the return of the movable component towards its initial position allows the control member to move behind the abutment surface under the action of the control means, thereafter to block further axial movement of the movable component towards its shifted position.

In the mechanism of this invention, there may be means to maintain the control member in its first position. That means could include a spring arranged to urge the control member in a direction away from its second position. Another possibility is to provide on the movable component a transversely-extending control surface on to which the control member is urged by a spring, that control surface maintaining the control member at its first position. For example, the control surface could comprise a ramp surface inclined towards the guide so that the control member is driven down the ramp surface to engage the guide, or could have a detent with which the control member is engaged under the action of a spring.

Once the control member has moved through said predetermined distance from its first position towards its second position, the control member is urged towards its second position, where it may block the movement of the movable component upon the return of the latter to its initial position. A spring may be provided for this purpose which spring could act directly on the control member. For example, in the case of a circularly-arranged mechanism, a helical compression spring could be employed which spring applies a torsional force to the control member, as well as an axial force thereon.

The predetermined distance through which the control member must move may be defined by a further surface on the movable component which further surface is inclined towards the second position of the control member and joins the first-mentioned control surface which maintains the control member at its first position. Then, moving the control member from the first-mentioned surface to the further surface will thereafter urge the control member to its second position.

Another possibility is for the movable component to have first and second parts between which the control member is located, the first and second parts being relatively movable in the direction of sliding movement of the movable component. The first part may be arranged as an actuator which bears on the control member, which first part has a cam surface engageable with the control member to drive the control member towards its second position once the control member has been moved through said predetermined distance. The cam surface of the actuator may engage the moving camming part of the control member, following movement of the control member through said predetermined distance, so as thereafter to urge the control member to its second position.

Preferably, the static camming part, which co-acts with the moving camming part on the control member to move that member through said predetermined distance, is formed on the guide. The location of the static camming part on the guide may be selected to govern said pre-set distance through which the movable component has to move from its initial position towards its shifted position before the control member is moved through said predetermined distance, whereafter blocking of the movable component will occur on returning the movable component towards its initial position. For example, the static camming part may be arranged at the end of the guide nearer the initial position of the control member, in which case triggering of the mechanism will occur after the movable component has moved a relatively short distance. Conversely, the static camming part may be arranged at the other end of the guide, in which case the mechanism will be triggered only after the movable component has moved through almost its full permitted range of movement towards its shifted position.

The latter arrangement may be advantageous when the mechanism is arranged as a safety medical needle assembly. In this case, the needle may be permitted to project from the static component (formed as a sleeve within which the needle normally is contained) by an amount sufficient to permit the filling of a connected syringe, but insufficient to trigger the mechanism. Subsequently, on using the syringe to perform an injection, the needle will be moved to project by the fullest amount from the sleeve, the mechanism then being triggered so that following the performance of an injection, the mechanism blocks subsequent movement of the sleeve relative to the syringe and needle, to maintain needle-protection.

With a safety needle assembly of this invention, the movable component may have a needle permanently secured thereto, a syringe being connectible to the movable component for communication with the needle. Alternatively, the movable component may be configured to accept a conventional medical needle, the hub thereof being received in the movable component. A further possibility is for the assembly to be configured for use with a pre-filled syringe having a needle permanently secured thereto, by fitting assembly of the invention to the syringe and needle combination, thereafter to give protection thereto.

This invention extends to injection apparatus whenever comprising a safety medical needle assembly of this invention in combination with a syringe connected thereto, to perform an injection or similar medical procedure.

By way of example only, several specific embodiments of latching blocking mechanisms and also of safety medical needle assemblies and medical syringes incorporating such assemblies, all arranged in accordance with this invention will now be described in detail, reference being made to the accompanying drawings, in which:

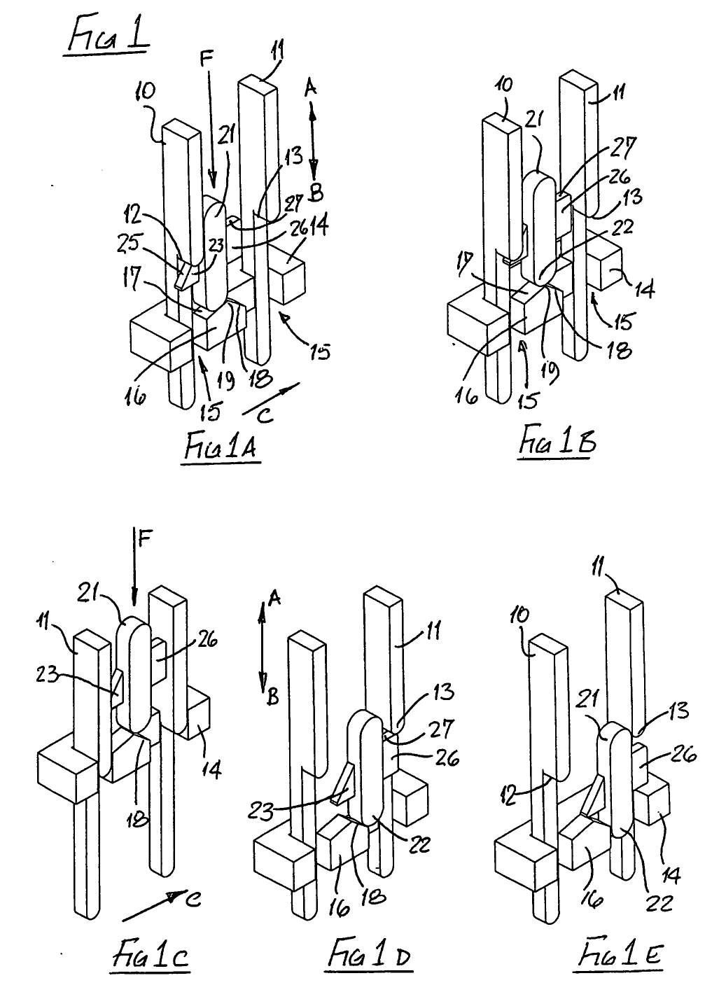

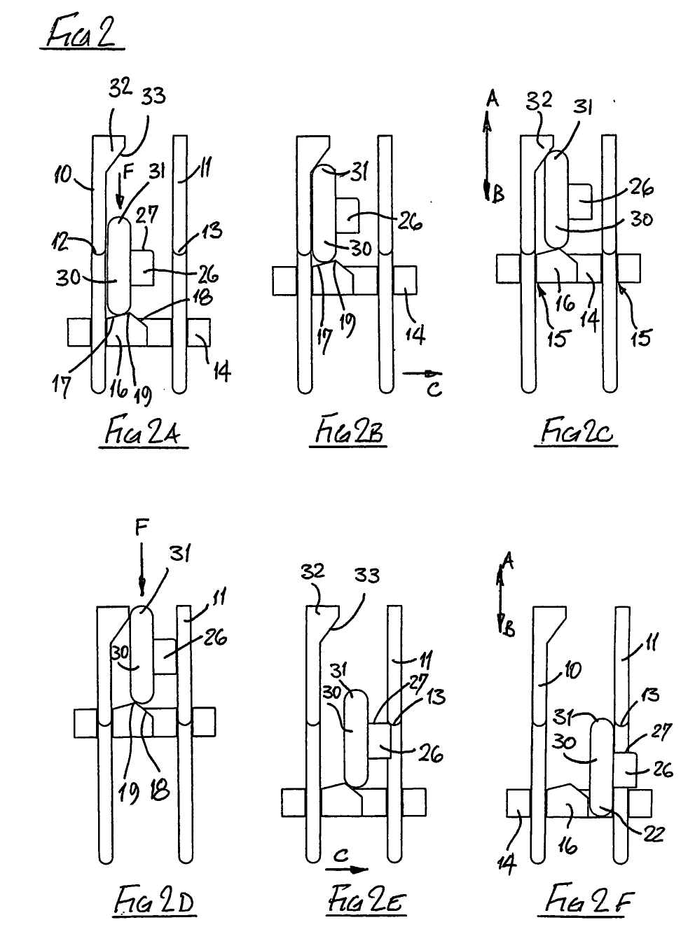

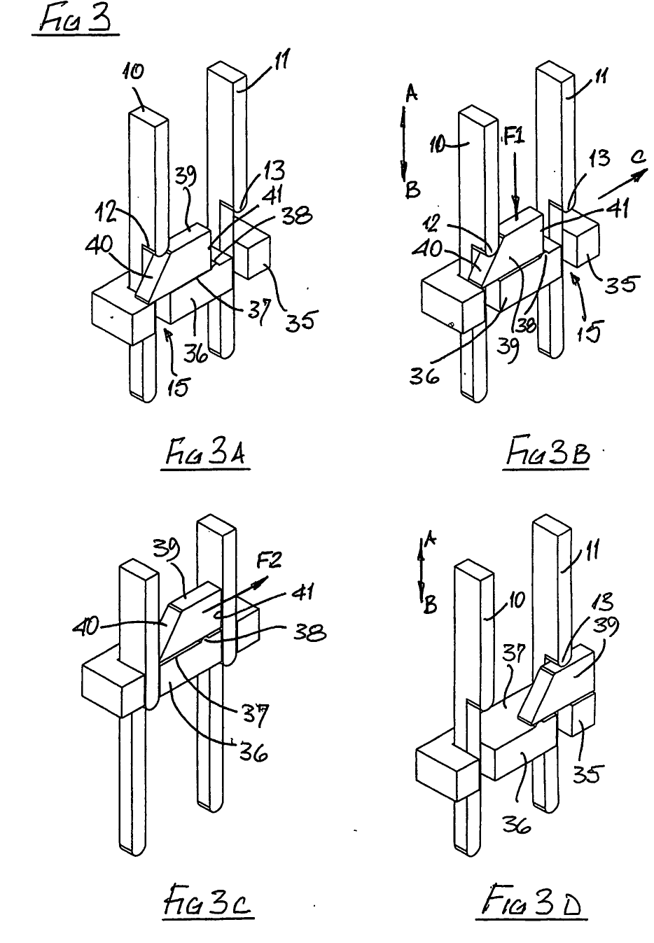

FIG. 1 is a diagrammatic isometric illustration of a linear acting first mechanism, wherein FIGS. 1A to 1E show the successive steps of the operation of the mechanism, from an initial setting to a latched and blocked setting;

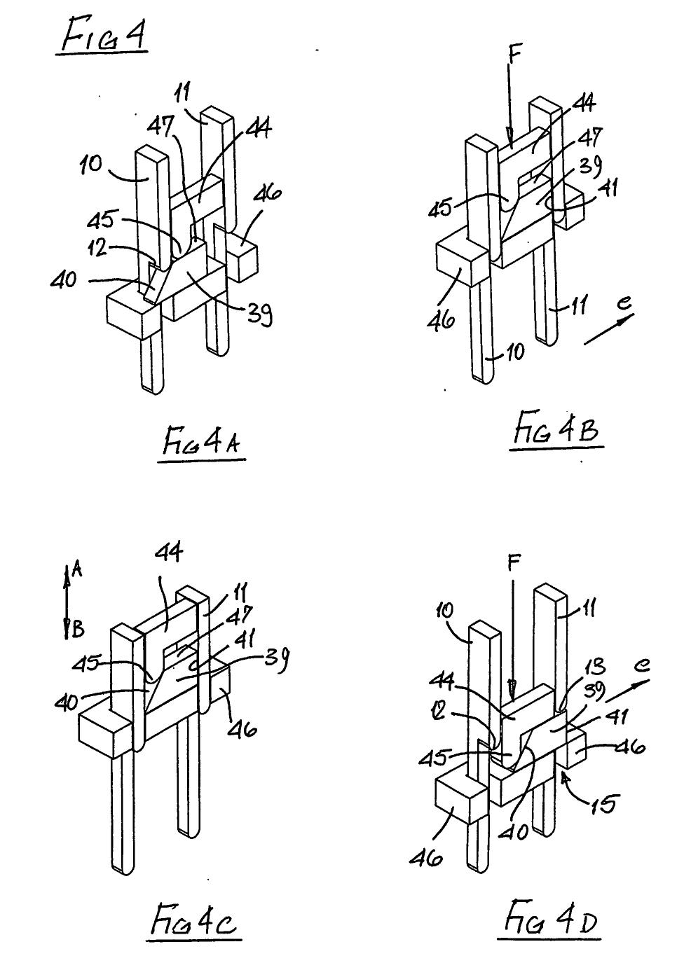

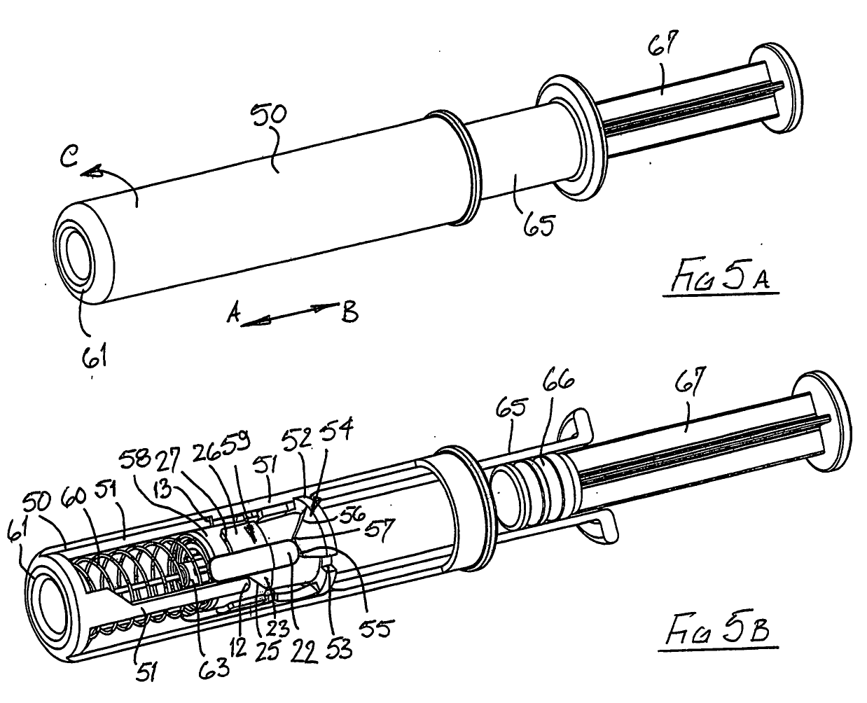

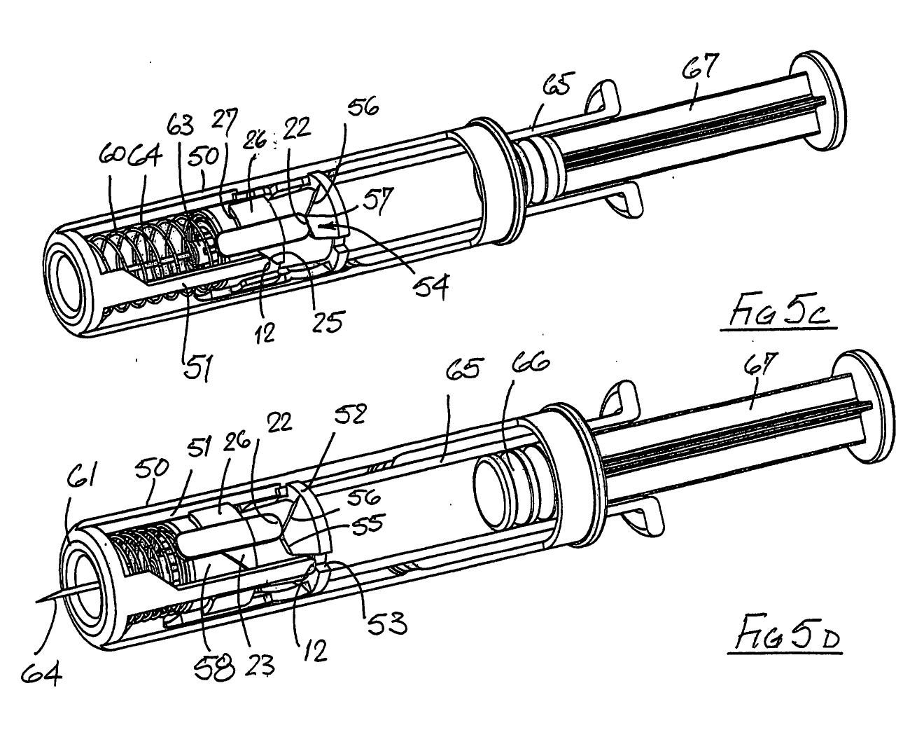

FIG. 2 is a diagrammatic front view illustrating a linear acting second mechanism, wherein FIGS. 2A to 2F show the successive steps of the operation of the mechanism, from an initial setting to a latched and blocked setting;

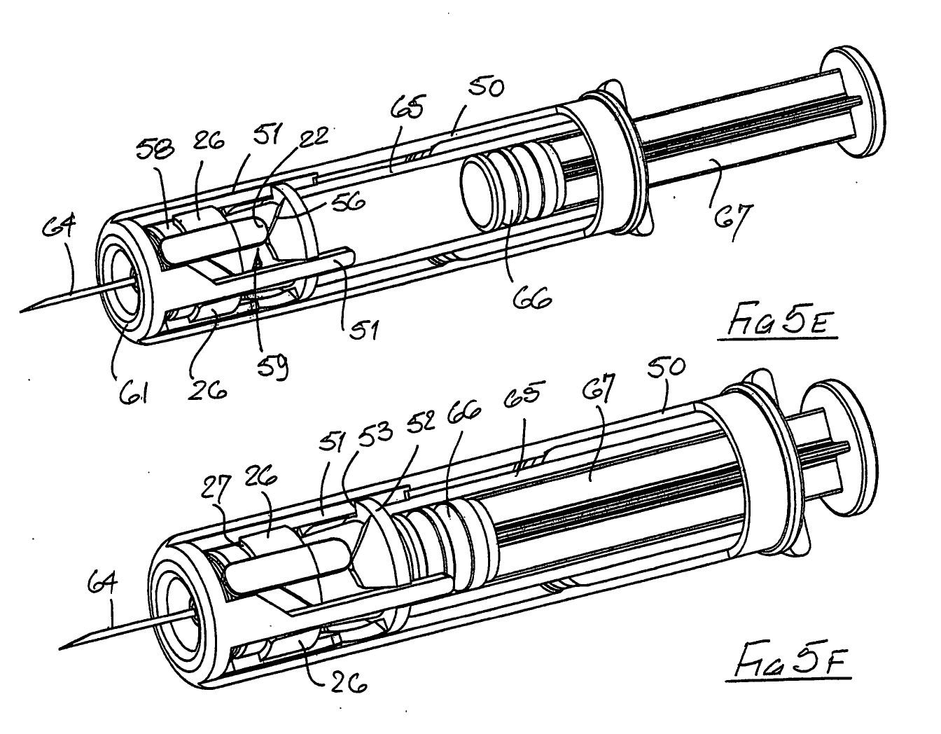

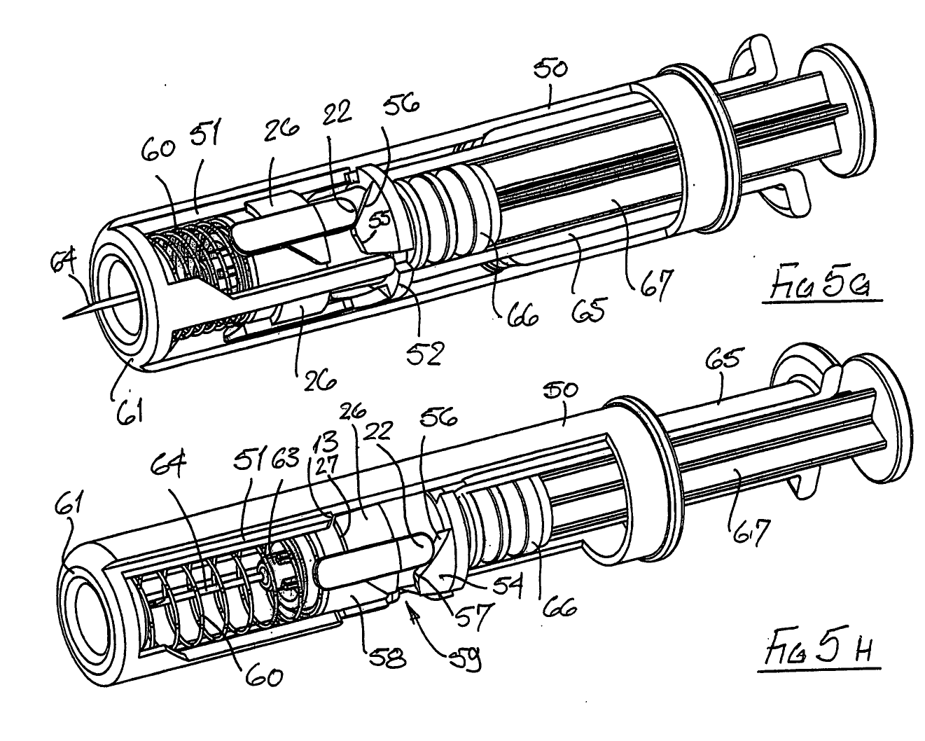

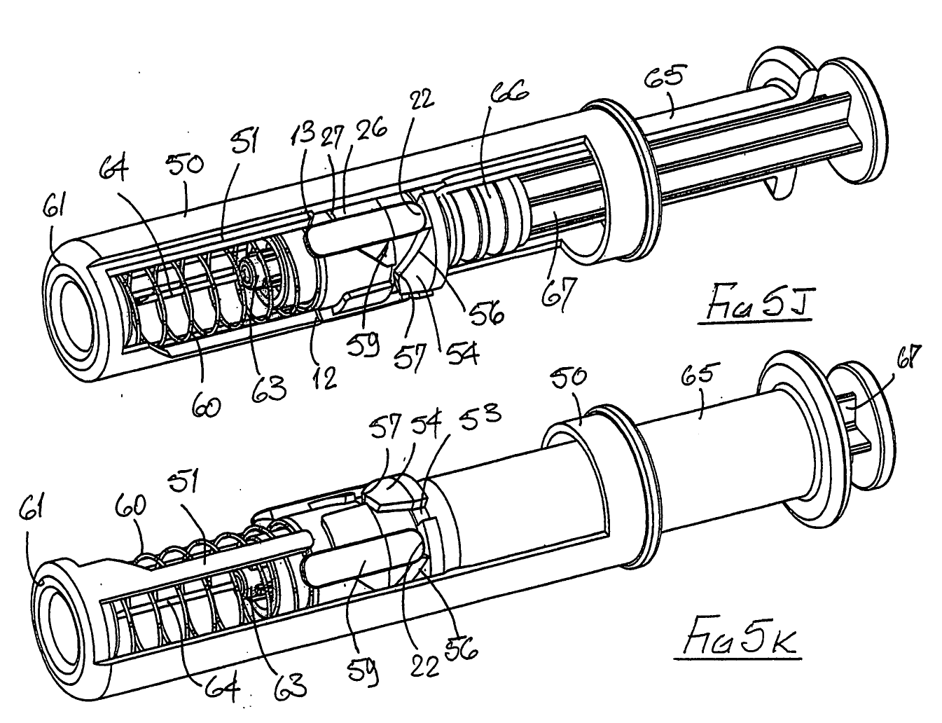

FIG. 3 is a diagrammatic isometric illustration of a linear acting third mechanism, wherein FIGS. 3A to 3D show the successive steps of the operation of the mechanism, from an initial setting to a latched and blocked setting;

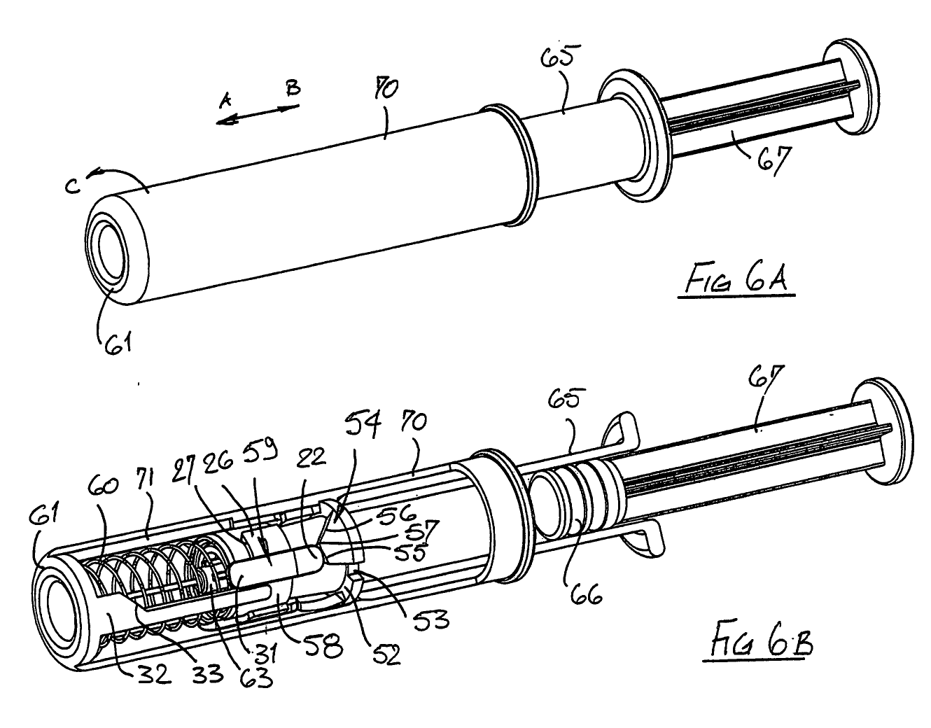

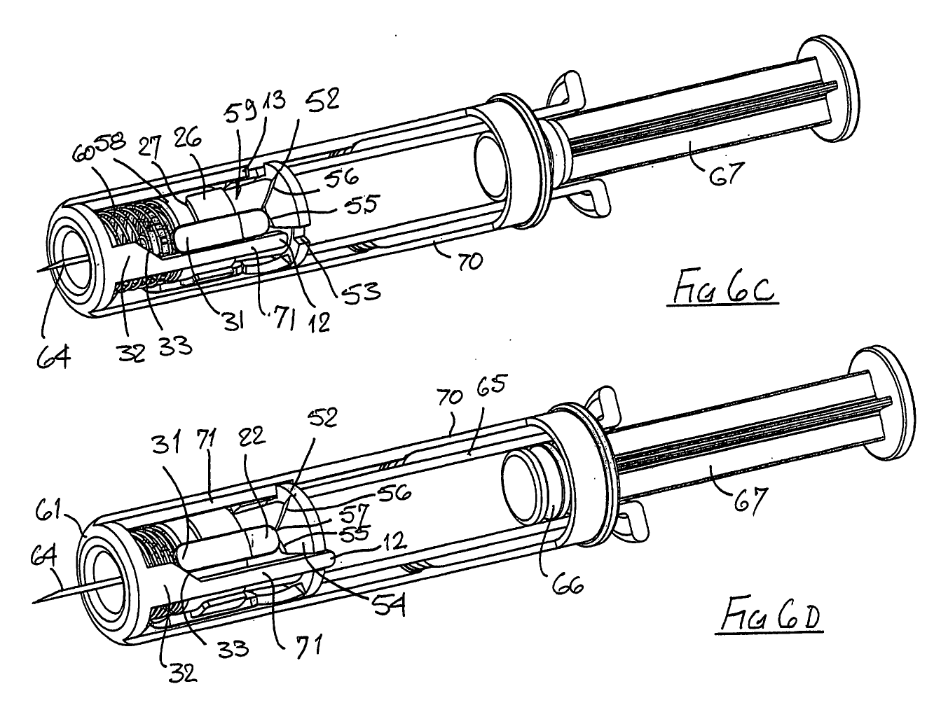

FIG. 4 is a diagrammatic isometric illustration of a linear acting fourth mechanism, wherein FIGS. 4A to 4D show the successive steps of the operation of the mechanism, from an initial setting to a latched and blocked setting;

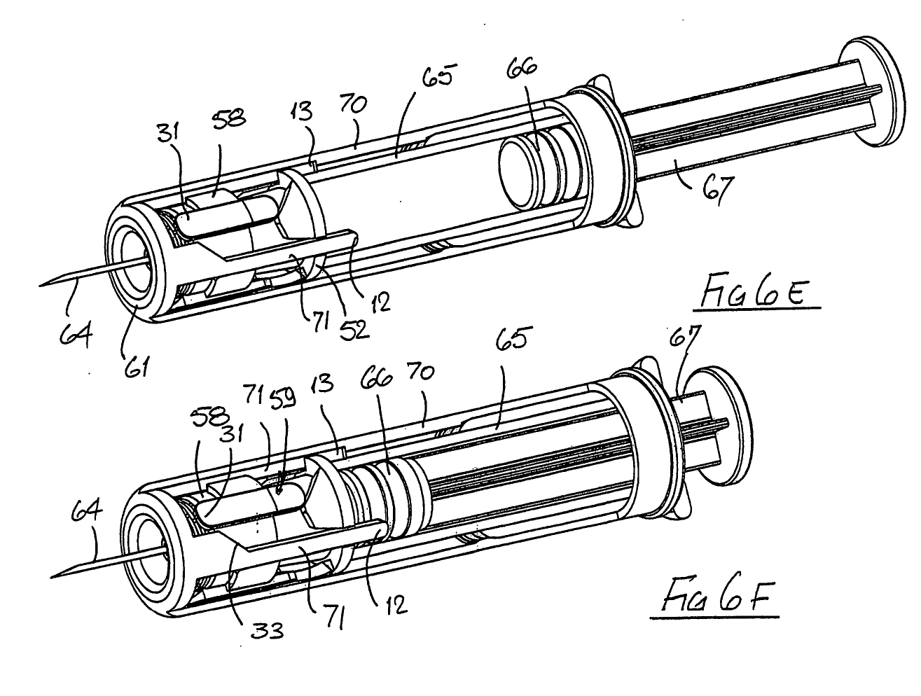

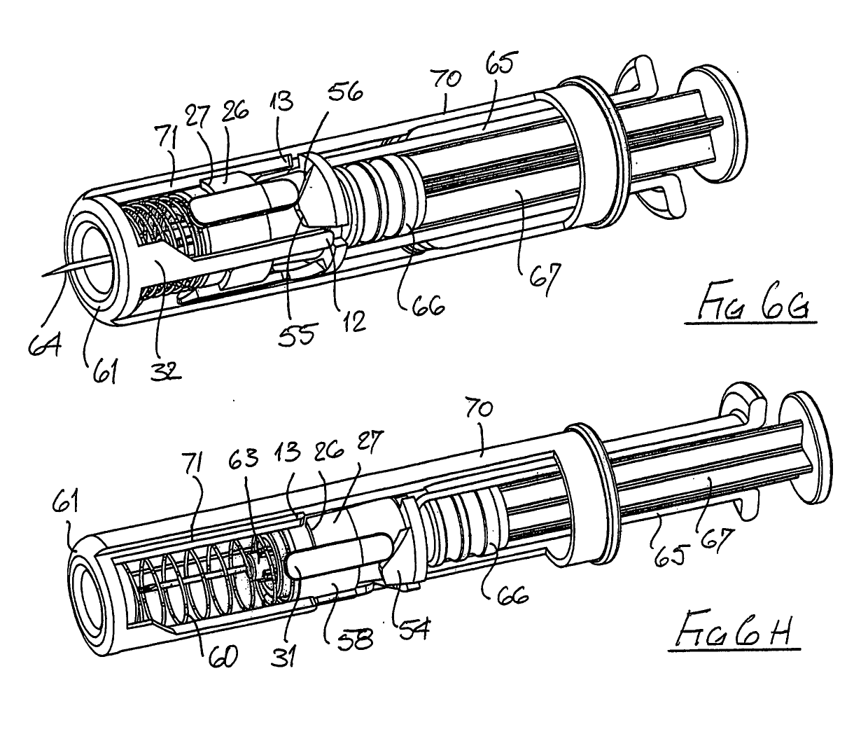

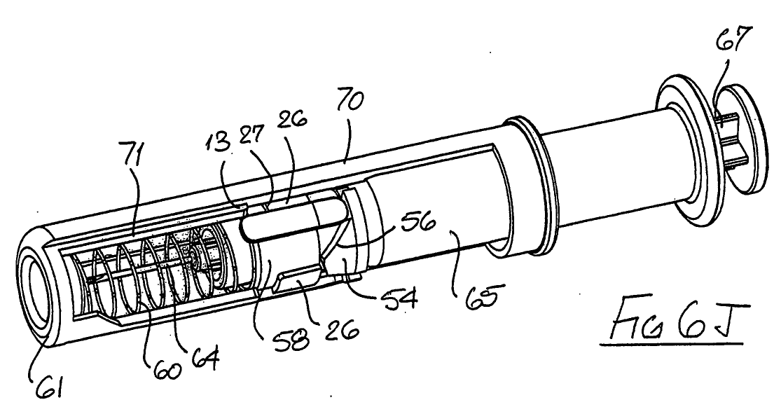

FIGS. 5A to 5K is a series of isometric illustrations of a first safety needle assembly associated with a syringe, but with various parts cut away for clarity, which needle assembly operates on the same principle as the mechanism of FIG. 1, and wherein FIG. 5A shows the complete assembly ready for use and FIGS. 5B to 5K show the successive steps of the operation of the mechanism, from an initial setting to a latched and blocked setting;

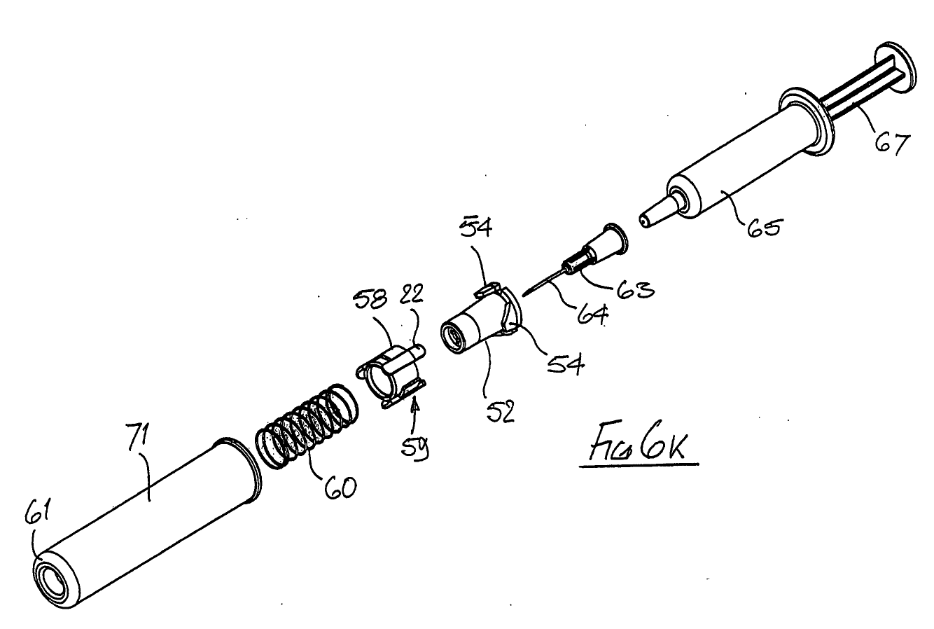

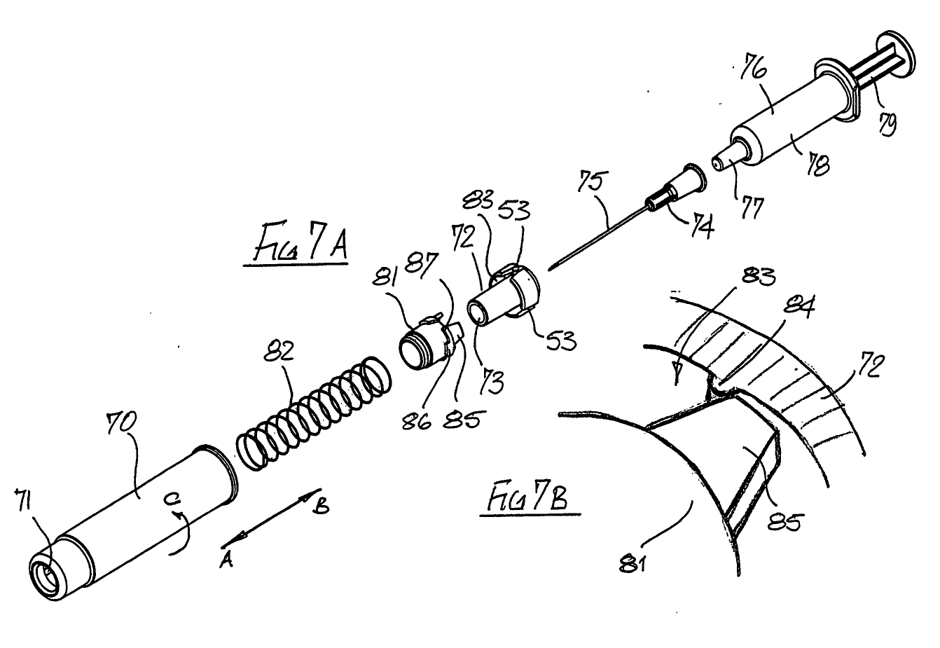

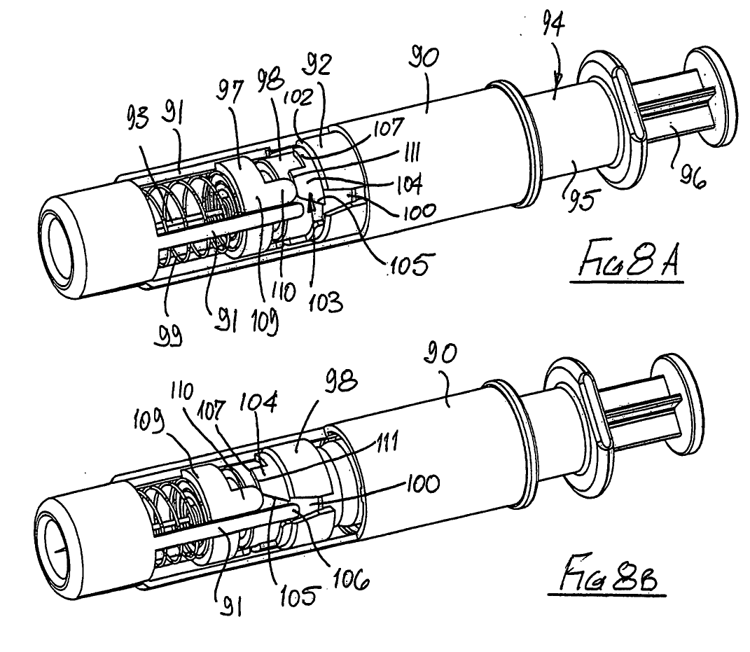

FIGS. 6A to 6K is a series of isometric illustrations of a second safety needle assembly associated with a syringe, but with various parts cut away for clarity, which needle assembly operates on the same principle as the mechanism of FIG. 2, and wherein FIG. 6A shows the complete assembly ready for use, FIGS. 6B to 6J show the successive steps of the operation of the mechanism, from an initial setting to a latched and blocked setting, and FIG. 6K is an exploded view of the components of this assembly;

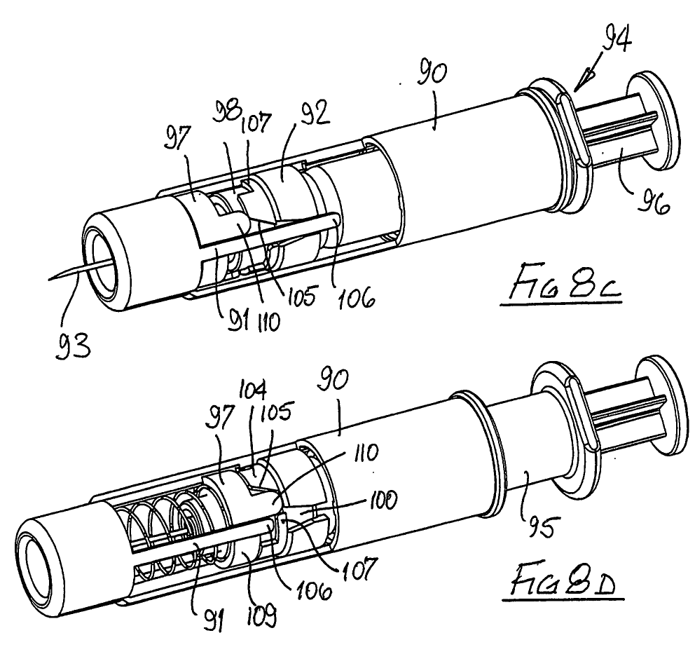

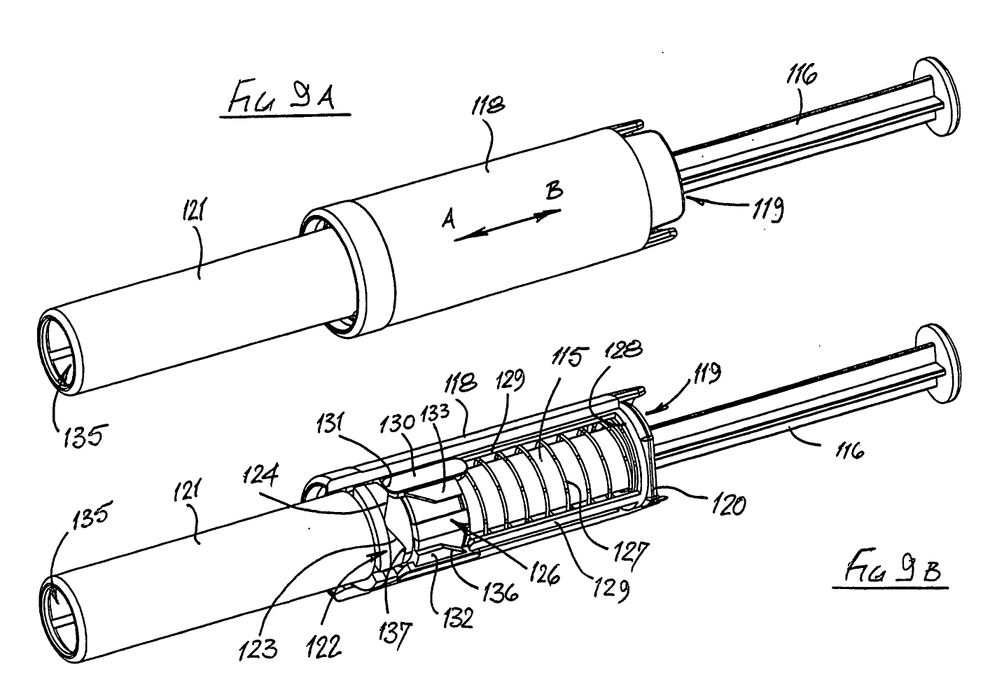

FIGS. 7A and 7B show a third safety needle assembly associated with a syringe and which operates on the same principle as the mechanism of FIG. 3, wherein FIG. 7A is an exploded view of the assembly, and FIG. 7B is a detailed view of an enlarged scale of a part of the assembly;

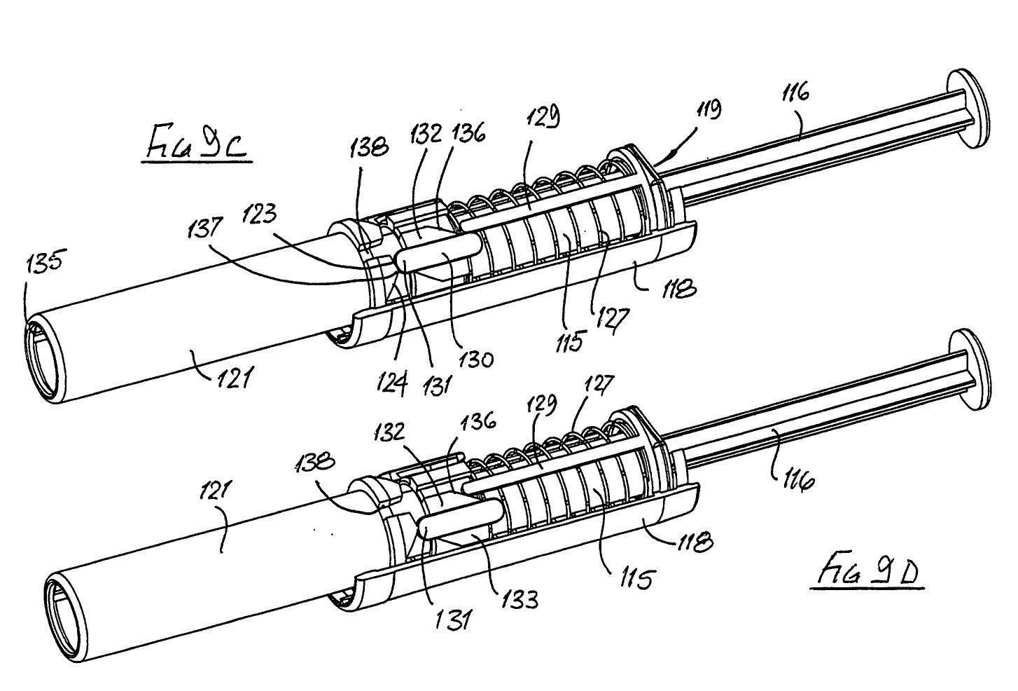

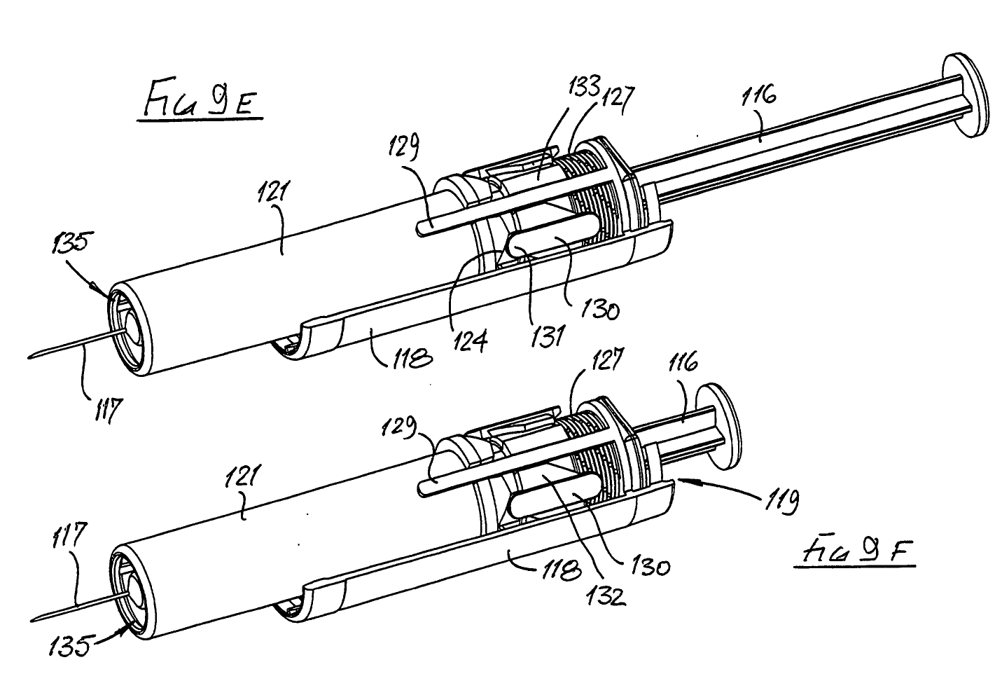

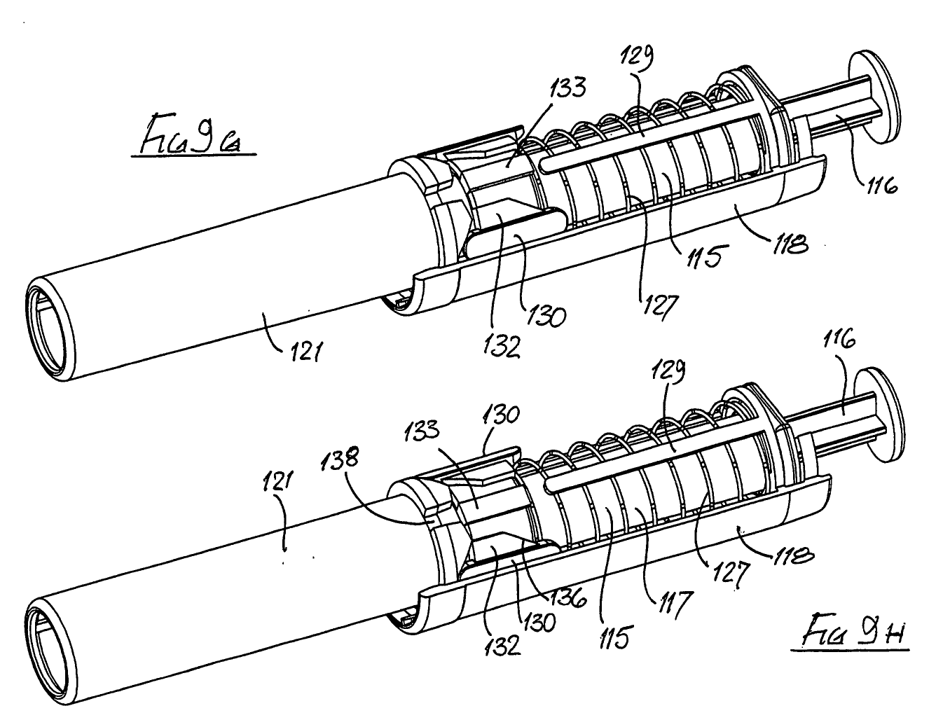

FIGS. 8A to 8D are isometric illustrations of a fourth safety needle assembly associated with a syringe, but with various parts cut away for clarity, which needle assembly operates on the same principle as the mechanism of FIG. 4, and wherein FIGS. 8A to 8D show the successive steps of the operation of the mechanism, from an initial setting to a latched and blocked setting;

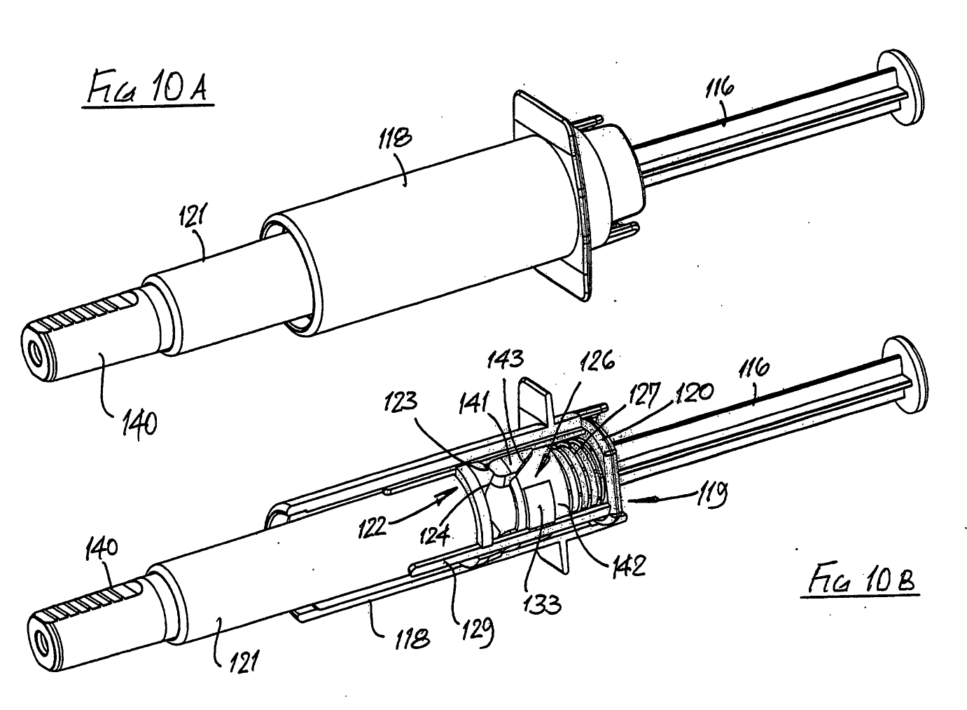

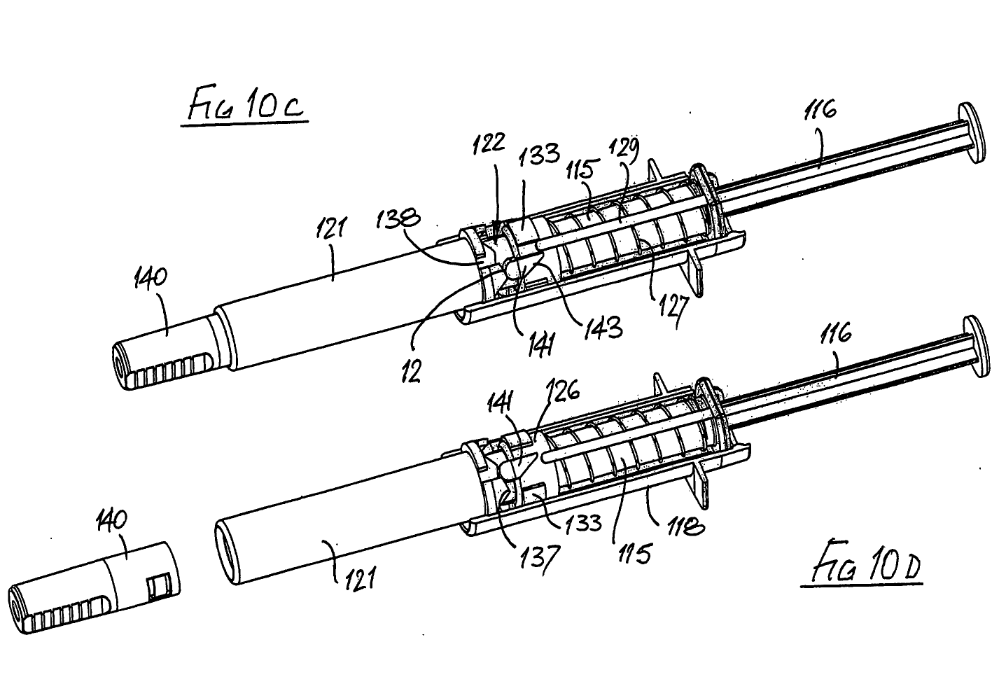

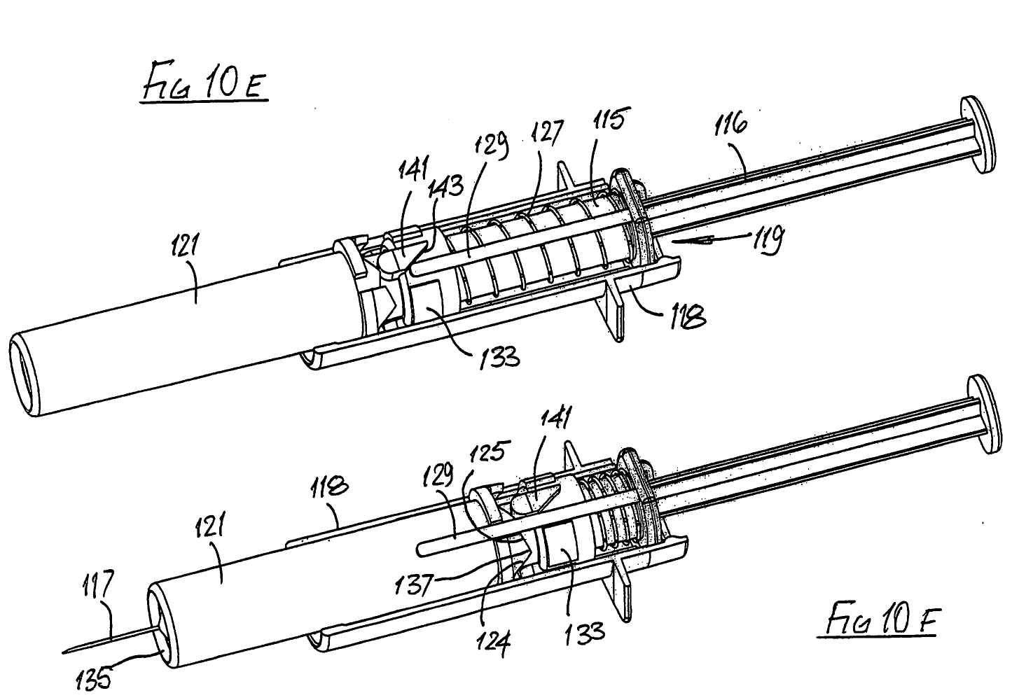

FIGS. 9A to 9H are isometric illustrations of a first embodiment of passive safety needle assembly for use with a pre-filled syringe, with various parts cut-away for clarity, wherein FIGS. 9A to 9H show the successive steps of the operation of the assembly from an initial setting to a latched and blocked setting; and

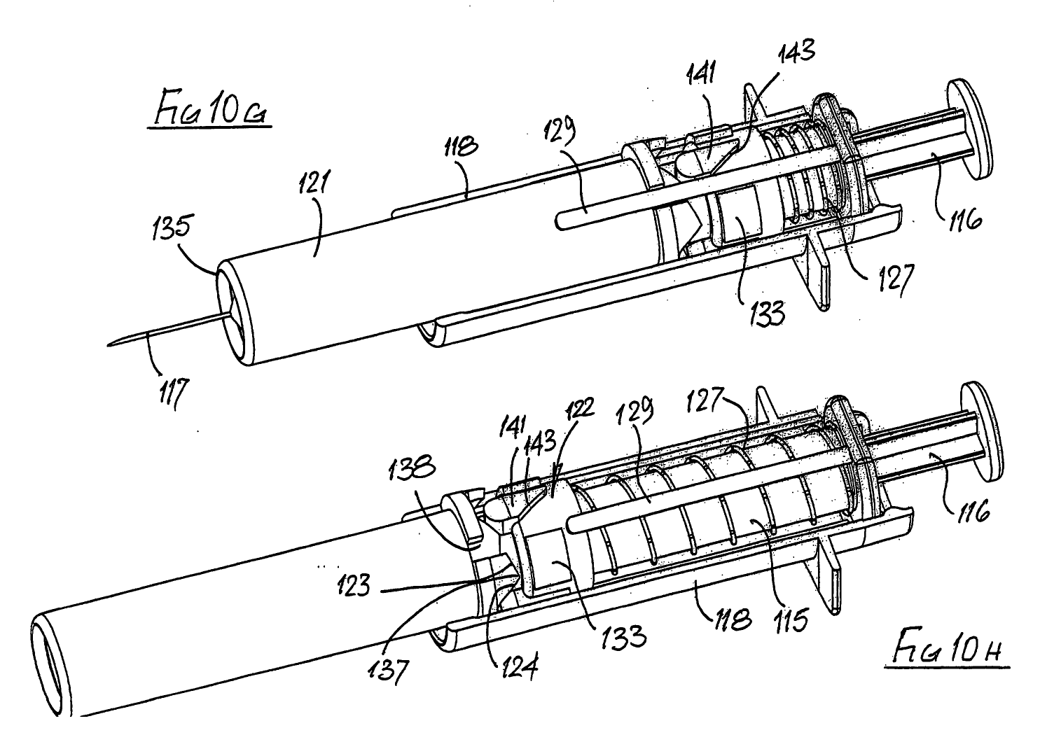

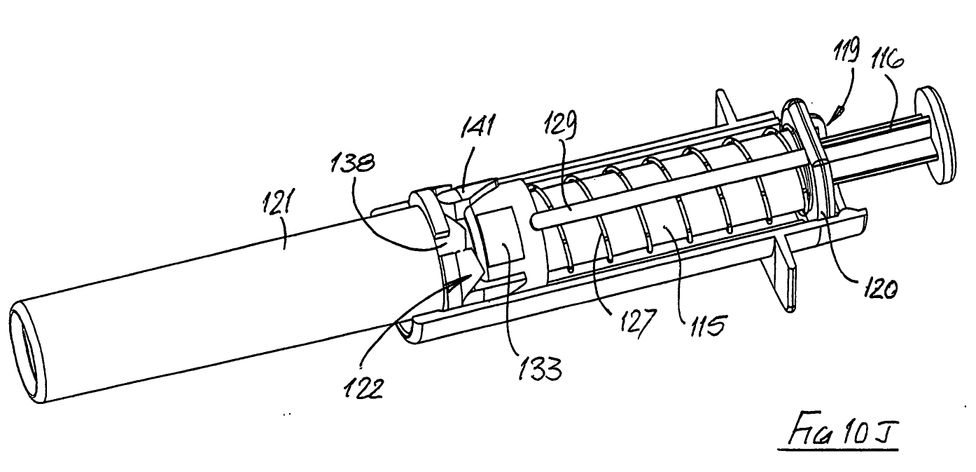

FIGS. 10A to 10J are isometric illustrations of a second embodiment of safety needle assembly for use with a pre-filled syringe, being a modification of the first embodiment of FIGS. 9A to 9H and again showing the successive steps of the operation of the assembly.

It is to be noted that the capital letter ‘I’ has not been used as a suffix in designating Figure numbers, in order to avoid confusion with the numeral 1.

Throughout the various embodiments of this invention, insofar as is possible the same reference numbers are used to designate the same components of the different embodiments, or closely similar components having essentially the same function. Such components will be described at the first occurrence, but then will not be described again in detail thereafter.

In the descriptions of these embodiments, the terms “forward” and “forwardly” respectively refer to the end of the mechanism to which the movable component is moved from its initial position and the direction of that movement, and the terms “rear” and “rearwardly” respectively refer to the other end of the mechanism and the direction towards that other end. Thus, in the case of a medical safety needle mechanism (FIGS. 5 to 10), the forward end is that end which is presented to a patient for performing an injection. Movement in the forward direction corresponds to movement in the direction of arrow A marked on the various drawings and movement in the rearward direction corresponds to movement in the direction of arrow B. Further, the transverse or circumferential direction (depending upon the embodiment) corresponds to the direction of arrow C, also marked on the drawings.

Referring initially to FIG. 1, there is shown a first embodiment of latching blocking mechanism of this invention, and which has the function of permitting a movable component to perform one cycle of operation, of sliding movement forwardly and then back to the initial position, whereafter further sliding movement of that movable component is blocked. The mechanism is arranged linearly—that is to say, both the sliding movement of the movable component is linear, and so too is the movement of a control member with respect to the movable component, but at right angles to the direction of sliding movement of the movable component.

In FIG. 1, there is shown a pair of parallel guides 10,11 each of which comprises a rail having a shoulder 12,13 formed partway therealong. A movable component 14 is in the form of a bar having slots 15 within which are received the guides 10,11, so that the component 14 is constrained for linear sliding movement along the length of the guides 10,11. The initial position of component 14 is shown in FIG. 1A; from there, the component may slide forwardly in the direction of arrow A to the position shown in FIG. 1C, and then rearwardly in the direction of arrow B back to its initial position, shown in FIG. 1E.

The central region of the movable component is formed with a control part 16 having first and second control surfaces 17,18, meeting at an apex 19. A control member 21 bears on the movable component 14, for sliding movement therewith along the length of the guides 10,11, by virtue of the interengagement of the rounded rear end 22 of the control member 21 with the control part 16 of that component 14. To one side of the control member 21 there is a lateral projection 23 the upper surface of which defines a cam face 25 engageable with shoulder 12 of the adjacent guide 10, on forward movement of component 14. On the opposite side of the control member 21 there is a blocking member 26 the forward surface 27 of which is engageable with the shoulder 13 of guide 11 when the control member has been moved laterally in the direction of arrow C, from its initial position shown in FIG. 1A. A spring (not shown) is arranged to apply a spring force F to the control member in the direction of arrow B and so into engagement with the movable component 14. As such, the spring force also urges the movable component in the direction of arrow B.

The operation of the mechanism of FIG. 1 will now be described. In FIG. 1A, the movable component 14 and the control member 21 are in their respective initial positions, urged to those positions by the spring force F. The rear end 22 of the control member 21 bears on the first control surface 17 of the control part 16 and so the control member is urged to the left (in the drawings, in the opposite direction to arrow C) to bear on guide 10. On the movable component 14 starting to be slid forwardly in the direction of arrow A, cam face 25 of the control member 21 comes into engagement with shoulder 12 of guide 10, and continued movement of the component 14 will cause the control member 21 to move in the direction of arrow C, as shown in FIG. 1B. As the control member moves in that direction, its rear end 22 rides up the first control surface 17 of the control part 16. When the lateral projection 23 moves clear of the shoulder 12 and so is located against the side of guide 10 (FIG. 1C), the control member has moved sufficiently in the direction of arrow C for the rear end 22 to ride over the apex 19 and then lie at the top (most forward part) of the second control surface 18. Further movement of the control member in the direction of arrow C is inhibited by the blocking member 26 bearing against the side of guide 11.

The guides 10,11 should have a sufficient length for the intended purpose of the mechanism such that the movable component 14 may have a sufficient range of movement for that purpose. When the movable component has reached its required furthest forward position, its direction of movement is reversed, to bring it back to its initial position. On moving sufficiently far in the direction of arrow B, from the position of FIG. 1C to that of FIG. 1D, the forward surface 27 of the blocking member 26 becomes aligned with the shoulder 13 on guide 11 whereafter the control member 21 may pass under shoulder 13, in the direction of arrow C. The spring force F acting on the control member will cause the control member to run down the second control surface 18 (FIG. 1D) so moving the blocking member 26 between the movable component 14 and shoulder 13 (FIG. 1E). Further, the rear end 22 of the control member will come off the second control surface 18 and lie alongside the control part 16, so preventing the control member moving in the direction opposed to that of arrow C, though there would be sufficient latching of the control member were it to remain on control surface 18.

As shown in FIG. 1E, the blocking member 26 will prevent the movable component 14 moving forwardly again in the direction of arrow A beyond the position where the forward surface 27 of the blocking member 26 engages shoulder 13 on guide 11. Further, the control member 21 cannot move back to its initial position as it lies alongside the control part 16. As such, the mechanism is now latched in a blocked setting where movement of the movable component 14 is inhibited, beyond such clearance as there may be between the final position of the movable component and the engagement of the blocking member forward surface 27 with shoulder 13.

It will be appreciated that the movable component 14 may be moved in the direction of arrow A from the position shown in FIG. 1A through a certain distance before the rear end 22 of the control member 21 rides over the apex 19 of control part 16. The mechanism may thus be tuned by adjusting the position of the projection 23 on the control member as well as the position of the shoulder 12 on guide 10, to permit reciprocation of the movable component 14 through a pre-set distance without the mechanism being triggered thereafter to block further forward movement of the movable component, on returning that component to its initial position.

The second embodiment is shown in FIG. 2, and is broadly similar to that of FIG. 1 but differs in the way in which the control member is moved laterally, to have the rear end 22 thereof ride over the apex 19 of the control part 16. In the embodiment of FIG. 2, the control member 30 is not provided with a lateral projection (unlike control member 21); rather, the camming action which moves the control member 30 laterally in the direction of arrow C is between the rounded forward end 31 of the control member and a lateral projection 32 provided at the forward end of guide 10. The lateral projection 32 provides a cam surface 33 with which the forward end 31 of the control member 30 is brought into engagement as the movable component 14 moves forwardly (FIG. 2B). Movement of the component 14 fully forwardly (FIG. 2D) brings the blocking member 26 into engagement with the side of guide 11, the rear end 22 then being at the top of the second control surface 18. On returning the movable component 14 back to its initial position (FIG. 2F) the blocking member moves between shoulder 13 of guide 11 and the movable component, as with the embodiment of FIG. 1. Similarly, the control member 30 is latched in this position, since the rear end 22 thereof moves alongside the control part 16.

In the embodiment of FIG. 2, the movable component 14 must be moved almost to its fully forward position before the mechanism is triggered. Thus, the component 14 may be cycled through a relatively large range of movement without becoming blocked and it is only when the position of FIG. 2D is reached that the movable component will thereafter be blocked, on returning that component to its initial position.

FIG. 3 shows the third embodiment of this invention which, whilst generally similar to those of FIGS. 1 and 2, differs in significant details. In FIG. 3, the movable component 35 has a control part 36 which defines a generally planar forward-facing control surface 37 between its slots 15 which receive the guides 10,11. Extending part-way across the control surface 37 is a rounded ridge 38. The control member 39 is generally wedge-shaped and has a cam surface 40. Spring forces F1 and F2 act on the control member 39, respectively in the direction of arrow B to engage the control member with the movable component 35 and in the direction of arrow C.

The initial position is shown in FIG. 3A, where cam surface 40 is adjacent shoulder 12 and the opposite end 41 of the control member is urged against ridge 38 by spring force F2. On moving the movable component 35 in the direction of arrow A, the shoulder 12 cams the control member 39 in the direction of arrow C, so forcing the control member to ride over the ridge 38 (FIG. 3B). The spring force F2 then brings end 41 of the control member to bear against the side of guide 11 (FIG. 3C), while the movable component continues its movement forwardly. On returning the movable component 35 back to its initial position (FIG. 3D) the spring force F2 acting on the control member 39 moves end 41 of the control member between shoulder 13 of guide 11 and the movable component 35. Thus, the control member serves to block further forward movement of the movable component in the direction of arrow A and the control member is maintained in that position by the spring force F2 acting thereon in the direction of arrow C.

FIG. 4 shows a modification of the arrangement of FIG. 3. Here, the movable component is in two parts, the forward part being in the form of an actuator 44 slidably disposed between the guides 10,11 and having a foot 45 which acts on the control member 39. The movable component 46 differs from that of FIG. 3, in that no ridge is furnished on the component 46 of this embodiment. Further, the control member 39 is not spring urged in the direction of arrow C; a spring force F acts solely on the actuator 44, in the direction of arrow B, so urging both the control member 39 and the movable component 46 in that direction.

The initial position is shown in FIG. 4A. Here, foot 45 of actuator 44 acts on the planar transverse upper surface 47 of the control member 39 and so imparts a force to that control member solely in the direction of arrow B; no transverse force in the direction of arrow C acts on the control member. On moving component 46 in the direction of arrow A, the control member is cammed in the direction of arrow C (FIG. 4B,4C), as with the third embodiment, until that member lies between the guides 10,11, at which point foot 45 of the actuator 44 bears on the cam surface 40 of the control member 39. Thus, the spring force F acting on the actuator 44 now will urge the control member 39 in the direction of arrow C.

On returning the movable component 46 to its initial position (FIG. 4D), that spring force F acts on the control member 39 to move the control member between the movable component 46 and the shoulder 13 of guide 11, so as thereafter to block further forward movement of the movable component, in the direction of arrow A. The continuing spring force F acting in the direction of arrow B on actuator 44 serves to maintain the control member 39 in the position of FIG. 4D.

Referring now to FIGS. 5 to 8, there are shown four safety needle mechanisms in conjunction with respective syringes, configured for the performance of injections. The safety needle mechanisms are respectively arranged generally in accordance with the principles of the four mechanisms described above with reference to FIGS. 1 to 4, though the safety needle mechanisms are in a circular form, unlike the linear mechanisms of FIGS. 1 to 4.

The safety needle mechanism of FIGS. 5A to 5K includes an outer sleeve 50 having three internally-formed equi-spaced axially-extending parallel guides 51, all of the same configuration. A movable component 52 is slidably disposed within the sleeve 50, that component having slots 53 which run along the guides and so restrain the movable component against rotation with respect to the sleeve. At the rearward end of the movable component, there are three control parts 54, disposed between respective pairs of guides 51, and each defining first and second control surfaces 55,56, an apex 57 being formed at the junction between those control surfaces. Both of those control surfaces 55,56 are helically inclined with respect to the axis of the sleeve 50, but in opposite senses, so as generally to correspond to the first and second control surfaces 17,18 of the first embodiment.

In the cut-away drawings of this embodiment, and also where relevant in the drawings of the fifth to eighth embodiments, the guides are shown as not extending from the forward end of the sleeve 50 back to the movable component 52, but this is just a consequence of the chosen cylindrical sectioning surface. This is co-incident with the internal plane of the guides rearwardly of the shoulders 12,13 provided therein. The ribs do in fact extend back to the rearward part of the sleeve, to be engaged by the respective slots 53 formed in the movable component.

Further, for convenience the mechanisms will be described solely with reference to the control part exposed at the cut-away region of the sleeve. There are in fact two further corresponding mechanisms distributed around the sleeve and operating in unison, making a total of three such mechanisms.

A cylindrical control member 58 is supported on a forward boss of the movable component 52, for rotation about the axis of the movable component, that control member having three separate control portions 59 each of which corresponds to the control member 21 of the first embodiment. Thus, each control portion 59 has a rounded rear end 22, a lateral projection 23 defining a cam face 25 and a blocking member 26. A helical compression spring 60 is disposed between an internally-directed flange 61 at the forward end of the outer sleeve 50 and the control member 58, to urge that member-rearwardly.

The movable component 52 has a bore in which is received the hub 63 of a medical needle 64, to which the forward tapered spigot of a syringe barrel 65 may connect. Within the syringe barrel 65, there is a piston 66 fitted with a plunger 67. The needle 64 may be a conventional needle used with a syringe, or may be permanently fitted within the movable component 52, in which case that component should be configured for connection to a standard taper lock arrangement as provided on a syringe.

The initial position is shown in FIGS. 5A and 5B, differing only in that the latter is partly cut away for clarity. A needle and syringe are shown connected to the movable component 52, with the needle projecting forwardly within the sleeve 50, but still wholly contained within that sleeve and so protected by it. The syringe barrel 65 also lies partly within the sleeve 50, and is movable relative thereto, forwardly in direction A, to project the needle from the front of the outer sleeve. The syringe is shown with its piston 66 withdrawn, ready to effect an injection of a medicament (not shown) already filled into the barrel 65. The control member 58 is urged rearwardly by the spring 60, such that the rounded rear end 22 of each control portion 59 bears on a corresponding first control surface 55 of the respective control part 54. As such, when viewed from the front, the control member 58 is urged in a clockwise direction (i.e. opposed to arrow C), such that each control portion bears on the adjacent guide 51, as shown in FIG. 5B.

In FIG. 5C, the sleeve 50 is shown as having moved rearwardly through a small distance with respect to the syringe barrel 65, the cam face 25 of the lateral projection 23 engaging shoulder 12 formed part-way along guide 51. This starts to cam the control member 58 so as to rotate in a counter-clockwise direction (i.e. the direction of arrow C). That camming action has been completed by the time the movable component 52 has moved sufficiently forwardly, to the position shown in FIG. 5D, the rear end 22 of the control member then bearing on the second control surface 56. By then, the needle 64 has started to project from the front end of the outer sleeve 50.

Continued forward movement of the syringe with respect to the sleeve allows the control member 58 to move fully forwardly (FIG. 5E), with the needle projecting by its greatest extent from the front of the sleeve. In this position, the spring 60 is compressed. Pressure on the plunger 67 moves the piston 66 forwardly within the syringe barrel so discharging the medicament therefrom (FIG. 5F).

Releasing pressure from the plunger allows the syringe barrel 65 to move rearwardly in the direction of arrow B with respect to the sleeve 50, under the action of spring 60 acting on the control member 58 and so also on the movable component 52. The blocking member 26 slides down the adjacent guide 51 (FIG. 5G) until the position shown in FIG. 5H is reached, where the forward surface 27 of the blocking member 26 becomes aligned with shoulder 13 of that guide, and may pass behind the shoulder. By then, the needle is once more wholly contained within the sleeve 50. It should be noted that in FIG. 5H the same part of the sleeve is shown cut away as in FIGS. 5B to 5G, though FIG. 5H is from a different angular view-point in order to show the interaction of the blocking member 26 with the adjacent guide 51.

Continued rearward movement of the syringe allows the rear end 22 of the control portion 59 to run down the second control surface 56, so further rotating the control member 58 in a counter-clockwise direction, with respect to the sleeve and movable component, in the direction of arrow C. This places the blocking member 26 between the shoulder 13 of guide 51 and the movable component (FIG. 5J). This position is also shown in FIG. 5K, but from a different viewpoint as compared to FIG. 5J.

In this setting, the blocking member 26 serves to block forward movement of the movable component 52 with respect to the sleeve 50 (or, conversely, rearward movement of the sleeve with respect to the syringe since the action is identical). As such, the needle 64 cannot be exposed once more and is held encapsulated within the sleeve 50. The entire unit may then be discarded, or in the case of a needle permanently mounted on the movable component 52, the syringe may be disconnected and the safety needle mechanism discarded.

In the foregoing description, reference has mainly been made to only one of the control portions 59 of the control member 58. All three control portions operate simultaneously and with the same functionality, and so any given guide 51 serves initially to define the position of a control portion of the control member 58, its shoulder 12 then camming the control member. At the completion of the operation of the mechanism, the shoulder of that guide serves to act with the blocking member 26 of another control portion. Thus, initially the shoulder on each guide serves as shoulder 12, and subsequently as shoulder 13.

The embodiment of FIG. 6 is similar to that of FIG. 5, but utilises the principles of the second embodiment (FIG. 2), rather than of the first embodiment. Externally, the mechanism as shown in FIG. 6A is indistinguishable from that of FIG. 5A but has a different function in that it is possible to move the sleeve 70 rearwardly with respect to the syringe (or the syringe and needle forwardly with respect to the sleeve) through a significant distance, and then to return the sleeve to its initial position, without the mechanism triggering to block a further cycle. This allows a syringe to be filled for example from a vial of medicament in preparation of the making of an injection and only when that injection is completed will the mechanism be locked out to prevent exposure of the needle once more.

As with the embodiment of FIG. 2, no lateral projection 23 is provided on any of the control portions 59 of the control member 58. Instead, the control member is rotated in a counter-clockwise sense by the interaction of the forward end 31 of each control portion 59 acting against a respective cam surface 33 formed on a projection 32 on each guide 51. In this way, the control member will be rotated counter-clockwise as the forward end 31 of the control portion 59 engages the cam surface 33 (FIG. 6D), so taking the rear end 22 over the apex 57 of the control part 54 (FIG. 6F). Following completion of the performance of an injection and the return of the movable component 52 to its initial position (FIG. 6H), the blocking member 26 moves between the movable component 52 and the shoulder 13 on the adjacent guide 51, so preventing a further cycle of operation.

In other respects, the operation of the embodiment of FIG. 6 is the same as that which has been described above with reference to FIG. 5 and so will not be described again here. This includes the provision of three control portions 59 all acting in unison.

FIGS. 7A and 7B show, in exploded and detail form respectively, another embodiment of safety needle assembly operating on essentially the same basis as the third embodiment of FIG. 3. The mechanism comprises an outer sleeve 70 provided with three internal guides 71 to restrain against rotation a movable component 72 slidably mounted within the sleeve. Each guide has a shoulder (not shown) formed part-way therealong (corresponding to shoulders 12,13) and the movable component has slots 53 in which the guides are received to hold the component against rotation with respect to the sleeve.

The movable component 72 has a bore 73 configured to receive and hold the hub 74 of a conventional needle 75. That hub 74 has a taper bore permitting the needle to be connected to a syringe 76 provided with a taper lock spigot 77 at its forward end. The syringe has a barrel 78 which is a free sliding fit within sleeve 70, and a plunger 79 connected to a piston within the barrel, for loading and then discharging the syringe.

A cylindrical control member 81 is rotatably supported on the movable component 72 and is urged rearwardly by a helical compression spring 82, acting between that control member 81 and the forward end of the sleeve 70. By virtue of the interengagement of the control member 81 with the movable component 72, that component also is urged rearwardly with respect to the sleeve 70, along with a needle and syringe connected thereto. The spring is torsionally pre-loaded and so applies a torque in a counter-clockwise direction to the control member 81.

The movable component 72 defines an annular channel 83 the base of which is provided with three equi-spaced ridges 84 (FIG. 7B). Each control portion of the control member 81 has three control surfaces 85 extending rearwardly, to bear on the base of the annular channel 83. When assembled, the spring 82 applies a torque in a counter-clockwise direction to the control member 81, so maintaining each control surface 85 in engagement with a respective ridge 84, the interaction between each control surface and the respective ridge preventing rotation of the control member.

Forwardly of the control surfaces 85, the control member 81 has three control portions each having a cam surface 86, engageable with a respective shoulder (not shown) formed on a guide 71 within the sleeve 70, as the assembly of the movable component and control member are moved forwardly. Sufficient forward movement will cause each control surface 85 to be driven over the respective ridge 84 of the movable component 72, but the control member cannot turn fully counter-clockwise when in this forward position, by virtue of the blocking portion 87 bearing against the next adjacent guide. However, on returning the movable component to its initial position, the control member is freed to turn counter-clockwise under the torsional action of the spring, so bringing the blocking portion 87 between the shoulder on the adjacent guide and the movable component. In this way the movable component is blocked against further forward movement.

The safety needle mechanism shown in FIGS. 8A to 8D utilises the principles of the fourth embodiment of FIG. 4. As with the previous embodiments, the safety needle mechanism includes an outer sleeve 90 defining three internal guides 91 to restrain against rotation a movable component 92 slidably mounted within the sleeve. The component 92 has a bore configured to receive the hub of a conventional medical needle 93. That hub has a taper bore permitting the needle to be connected to a syringe 94 provided with a taper lock spigot at its forward end (as shown in the exploded of FIG. 7A of the previous embodiment). The syringe has a barrel 95 which is a free sliding fit within sleeve 90, and a plunger 96 connected to a piston within the barrel, for filling and then discharging the syringe.

The movable component 92 carries a cylindrical actuator 97, a cylindrical control member 98 being rotatably supported by the movable component 92, between the rear part thereof and the actuator 97. A helical compression spring 99 disposed in the forward part of the sleeve 90 bears on the actuator 97 and so urges rearwardly the actuator 97, control member 98 and movable component 92. In this way, a connected syringe and needle are also urged rearwardly with respect to the sleeve 90.

The movable component 92 has three slots 100 in its periphery, in which are received the guides 91 to prevent rotation of that component 92 with respect to the sleeve 90. A circumferential shoulder 102 is provided on the component 92 which the cylindrical control member 98 bears. That control member has three control portions 103 each of which has a control wedge 104 with a cam surface 105 which co-operates with a shoulder 106 on the guide 91, corresponding to shoulders 12,13 on guides 10,11 of the first-described embodiments. Further, each control wedge includes a blocking portion 107 also adapted to co-operate with a shoulder of a guide, to block forward movement of the movable component, when the control member 103 has been turned sufficiently in a counter-clockwise direction, when viewed from the front.

The actuator 97 is also slidably received within the sleeve 90, but is restrained against rotation by portions 109 thereof which are received between the guides 91. Each portion 109 has a foot 110 projecting rearwardly and operating on a respective control wedge 104 of the control member 103.

The initial setting is shown in FIG. 8A; only one of the three control portions is here visible and will be described in the following. In this setting, foot 110 of the actuator 109 bears on a circumferential surface 111 of the control wedge 104 and the cam surface 105 of the control wedge is adjacent a shoulder 102 of a guide 91. As such, no torque is applied to the control member 103. On forward movement of the syringe and movable component 92 with respect to the sleeve 90 (or rearward movement of the sleeve 90 with respect to the syringe) drives the cam surface 105 over the shoulder 102 of the guide 91, so turning the control member 103 in a counter-clockwise direction (FIG. 8B). The foot 110 of the actuator 97 then moves on to the most forward part of the cam surface 105, such that the spring pressure on the actuator 97 also tends to turn the control member 103 in a counter-clockwise direction. However, that control member 103 cannot turn further in a counter-clockwise direction by virtue of the blocking portion 107 bearing against the next adjacent guide 91.

Continued relative movement between the outer sleeve 90 and syringe 94 causes the needle 93 fully to project from the sleeve 90 (FIG. 8C), the spring 99 becoming compressed within the forward part of the sleeve. The plunger 96 of the syringe may then be driven home to dispense medicament within the syringe, and on releasing the plunger, the syringe, movable component 92, control member 98 and actuator 97 will all move rearwardly with respect to the sleeve, under the action of the spring 99. As the initial setting of the movable component 92 is reached once more, the blocking portion 107 of the control member is moved behind guide 91 (FIG. 8D) by virtue of the spring force on the actuator 97 and its foot 110 bearing on the cam surface 105 of the control wedge. The foot may thus move rearwardly down the cam surface as the control member 98 rotates in a counter-clockwise sense, until the blocking member is wholly positioned between the shoulder on a guide and the movable component. In this position, the foot 110 prevents clockwise rotation of the control member, and the movable component 92 is blocked in the position of FIG. 8D with the needle 93 wholly contained within and encapsulated by the sleeve 90.

Though this embodiment has been described as being suitable for use with a conventional syringe and detachable needle, it particularly lends itself to use with a pre-filled syringe having a needle permanently secured thereto and which may be press-fitted into the movable component 92. Once an injection of the medicament in the syringe has been completed, the needle is made safe by the mechanism and the entire assembly, including the syringe and needle, is discarded.

Referring now to FIG. 9A to 9H, there is shown a pre-filled syringe having a glass body 115 defining a cylindrical chamber for a liquid medicament to be dispensed, the syringe having a plunger 116 fitted with a piston (not shown), for expelling the medicament pre-filled into the body 115 at the time of manufacture. The body has a hub at its forward end to which a needle 117 (FIGS. 9E and 9F) is permanently secured, which needle is protected by a sheath (also not shown) at the time of manufacture but which must be removed before fitting the syringe to the safety assembly of this embodiment.

The passive safety assembly comprises an outer sleeve 118 within which the body 115 is received from the rearward open end 119 thereof, that open end of the sleeve being internally profiled to engage with a flange 120 formed at the rearward end of the syringe body. In this way, once the syringe has been pushed fully into the sleeve, it is retained therein.

A movable component 121 is slidably received within the sleeve and also is slidable over the outer surface of the body 115 of the syringe. In its initial position shown in FIGS. 9A to 9C, the component 121 projects to its fullest extent from the outer sleeve 118 and so wholly encloses the needle 117. At its rearward end, the movable component 121 has three control parts 122, each having a first control surface 123 and a second control surface 124, which control parts and first and second control surfaces correspond to the control parts 54 and first and second control surfaces 55 and 56 of the embodiment described with reference to FIG. 5 of said application.

Also slidably mounted within the outer sleeve 118 is a control member 126, biased to the forward position shown in FIGS. 9B and 9C by a helical compression spring 127 acting between the control member and an annular abutment 128 formed within the rearward open end 119 of the body 115. By virtue of the control member 126 bearing on the movable component 121, that component 121 is also urged to its initial position, projecting to the greatest extent from the outer sleeve 118, as shown in FIGS. 9A to 9C.

Three parallel guides 129 are formed internally of the outer sleeve 118 at equi-spaced intervals and extend for the greater part of the length of the outer sleeve. The control member 126 is of a similar form to control member 58 of FIG. 5 of said application and has three bars 130 each having a rounded forward end 131 for cooperating with the first and second control surfaces 123 and 124. Associated with each bar 130 is a lateral projection 132 (corresponding to lateral projection 23 of FIG. 5) for cooperation with a respective guide 129 and a blocking member 133 (corresponding to blocking member 26 of FIG. 5) also for cooperation with a respective guide 129.

As will be appreciated, the arrangement of the control parts 122, the control member 126 and the guides 129 all have the same functionality as the corresponding parts 54, control member 58 and guides 51 of the embodiment of FIG. 5. As such, the operation of the mechanism will be described only briefly, here.

The initial setting of the assembly is shown in FIGS. 9A, 9B and 9C, FIG. 9C showing the components in the same relative positions as FIG. 9B but with more of the outer sleeve 118 cut away and also with the assembly turned through approximately 60° for the sake of clarity. The movable component 121 projects to the greatest extent from the outer sleeve 118, and is urged to that position by the spring 127 acting through the control member 126.

Though there are three identical mechanisms spaced around the assembly, the action of only one of those will be described in the following. The rounded forward end 131 of the bar 130 bears on the first control surface 123 of the movable component 121 but the control member cannot rotate in a counter-clockwise direction when viewed from the open forward end 135 of the movable component 121 furthest from the syringe, by virtue of the interaction of the other end of the bar 130 with an adjacent guide 129 (FIG. 9C). When an injection is to be performed, the open forward end 135 of the movable component is engaged with the injection site and on applying pressure by holding the outer sleeve 118 and moving it towards the injection site, the movable component 121 starts to move towards its withdrawn position within the outer sleeve 118, in the rearward direction of arrow B.

Almost immediately, the forward end of guide 129 engages the lateral projection 132 and, by virtue of the cam face 136 of that projection, the control member is turned in a clockwise direction, when viewed as aforesaid (FIG. 9D). This causes the rounded forward end 131 of bar 130 to ride over apex 137 between the first and second control surfaces 123,124 so that the control member 122 is now urged in a clockwise sense by the interaction of the bar 130 with the second control surface 124 (FIG. 9E). The turning of the control member in that sense is limited by the blocking member 133 engaging the next adjacent guide 129. As best seen in FIGS. 9D and 9E, a slot 138 is formed in the forward end of the movable component 121, to accommodate the guide 129 and prevent relative rotational movement between the outer sleeve 118 and the movable component 121.

When the movable component 121 has moved fully rearwardly to its withdrawn position (FIG. 9E) and the needle 117 projects to the greatest extent, the injection of the medicament is performed by depressing the plunger 116 (FIG. 9F), whereafter the entire assembly is moved away from the injection site. This allows the movable component 121 to return to its initial position under the action of spring 127 (FIG. 9G). Once the blocking member 133 comes free of the guide 129, the control member 126 is allowed to turn further in a clockwise sense as the rounded forward end 131 of bar 130 is urged to run down the second control surface 124, so bringing the blocking member 133 into alignment with the guide 129 (FIG. 9H). When in this position, the blocking member 123 will now prevent withdrawal movement of the movable component 121 in the direction of arrow B, thereby preventing the needle 117 being exposed for a second time.

FIGS. 10A to 10J illustrate a similar arrangement to that described above, but differing in certain respects which will be explained below. However, the basic operating principle of the assembly is essentially the same and so components having the same function as those of the previous embodiment are given the same reference characters and will not therefore be described in detail once more.

With the embodiment of FIG. 9, the mechanism is set ready to be triggered as soon as the movable component 121 starts its withdrawal movement in the direction of arrow B. With the embodiment of FIG. 10, the mechanism is initially unset and becomes set for triggering only on removing a cap 140 fitted into the forward end 135 of the movable component 121. This is advantageous when it is necessary to adjust the dose to be dispensed by the pre-filled syringe, before performing an injection.

The cap 140 is arranged to engage the hub end of the syringe within the mechanism, so as to hold the movable component 121 in the position shown in FIGS. 10A and 10B, and so partially in a withdrawn position. However, the needle remains protected by virtue of the cap 140. By manufacturing both the outer sleeve 118 and the movable component 121 from transparent materials, the syringe will be visible within those components and so the quantity of medicament within the syringe may also be seen. When the assembly is in the initial position of FIGS. 10A and 10B, the plunger may be partially depressed to expel the excess medicament from the syringe until the required quantity remains therewithin, as determined by viewing the position of the piston within the syringe body.

The cap 140 is then pulled in the direction of arrow A to come free of the syringe hub and to draw the movable component 121 in the same direction, so as further to project from the outer sleeve 118 (FIG. 10C). During this, the bar 141 of the movable component 121 slides down the adjacent guide 129 and eventually comes free of that guide, as the cap 140 comes away from the movable component 121. This allows the mechanism to be set, with a cam surface 143 formed on the rearward end of bar 141 presented to the forward end of a guide 129 (FIG. 10D). An injection may then be performed as described above with reference to FIG. 9, but during the initial stage of the movable component moving rearwardly in the direction of arrow B, the cam surface 143 on bar 141 turns the control member 142 in the counter-clockwise direction, to cause the rounded forward end of the bar to ride over the apex 137 between the first and second control surfaces 123,124 (FIG. 10E), as has been described above.

Continued withdrawal movement of the movable component exposes the needle 117 (FIG. 10F) but further rotation of the control member 142 is prevented by the blocking member 133 thereof engaging an adjacent guide 129. When the movable component 121 has withdrawn fully, so exposing the needle 117, an injection is performed by pressing the plunger 116 (FIG. 10G) and then the entire assembly is moved away from the injection site, so allowing the movable component 121 to move fully forwardly in the direction of arrow A (FIG. 10H). When the blocking member 133 comes free of guide 129, the control member is urged to turn further in a counter-clockwise sense by the rounded forward end of the bar 141 bearing on second control surface 124, so bringing the blocking member into alignment with the guide 129 (FIG. 10J). This blocks withdrawal movement of the movable component 121 for a second time, in the direction of arrow B.

1. A latching blocking mechanism, comprising:
a static component including an elongate guide;
a static abutment surface;
a movable component slidable along the guide between initial and shifted positions;
a control member arranged for movement with the movable component in the sliding direction thereof but displaceable with respect thereto in a direction transverse to the sliding direction of the movable component, the control member initially being disposed at a first position relative to the movable component;
control means arranged to urge the control member transversely towards a second position from the first position, which control means becomes active only after the control member has moved by a predetermined distance from its first position towards its second position; and
a static camming part co-operable with a moving camming part on the control member and arranged to move the control member through said predetermined distance from its first position on movement of the movable component from its initial position towards its shifted position;
whereby following the movement of the control member through said predetermined distance and the return of the movable component towards its initial position, the control member is driven by the control means to a blocking position between the static abutment surface and the movable component thereafter to block further movement of the movable component towards its shifted position.

2-29. (canceled)

30. A safety medical needle assembly, comprising:
a tubular component including an internal elongate guide;
an abutment surface formed on the tubular component;
a movable component disposed within the tubular component and arranged for axial sliding movement with respect thereto between initial and shifted positions, the movable component being adapted to receive the hub of a medical needle so that the needle projects within but is protected by the movable component when in its initial position;
a control member arranged for axial movement with the movable component but rotatable with respect to the movable component, the control member initially being disposed at a first position relative to the movable component;
control means arranged to urge the control member towards a second position angularly displaced from the first position, which control means becomes active only after the control member has turned through a predetermined angle towards the second position; and
a camming part on the tubular component co-operable with a moving camming part on the control member and arranged to turn the control member through said predetermined angle from its first position on axial movement of the movable component towards its shifted position to cause the needle to project from the movable component;
whereby the movement of the movable component towards its shifted position to cause the needle to project therefrom also causes the control member to turn through said predetermined angle whereafter the return of the movable component towards its initial position allows the control member to move behind the abutment surface under the action of the control means, thereafter to block further axial movement of the movable component towards its shifted position.

31. A safety medical needle assembly as claimed in claim 30, wherein the camming part on the tubular component comprises an abutment formed on the guide, and the camming part on the control member is engageable therewith to cause turning movement of the control member during an initial stage of the movement of the movable component from its initial position.

32. A safety medical needle assembly as claimed in claim 30, wherein the camming part on the tubular component comprises a projection formed on the guide and disposed at or adjacent the end thereof, for engagement by the camming part on the control member as the movable component approaches its shifted position, to cause turning movement of the control member.

33. A safety needle assembly as claimed in claim 30, wherein there is provided a helical compression spring acting between the tubular component and the movable component, to urge the movable component towards its initial position.

34. A safety medical needle assembly as claimed in claim 33, wherein the compression spring is pre-loaded to apply a torque to the movable component, to rotate the movable component towards its second angular position.

35. A safety medical needle assembly as claimed in claim 34, wherein there is provided initialization means including a control surface on the movable component and on to which the control member is urged by said spring.

36. A safety medical needle assembly as claimed in claim 35, wherein said control surface of the movable component is a ramp surface inclined towards the guide.

37. A safety medical needle assembly as claimed in claim 35, wherein said control surface of the movable component extends in a radial plane.

38. A safety medical needle assembly as claimed in claim 37, wherein said control surface of the movable component has a detent with which the control member is engaged under the action of the spring.

39. A safety medical needle assembly as claimed in claim 33, wherein the movable component has a further surface on to which the control member is urged by the spring, which further surface is inclined towards the second position of the control member and adjoins the control surface of the first control means.

40. A safety medical needle assembly as claimed in claim 39, wherein the junction between the control surface and the further surface defines the predetermined angle from the first position of the control member through which the control member is turned by the co-operable camming parts.

41. A safety medical needle assembly as claimed in claim 30, wherein the movable component has first and second parts between which the control member is located, the first and second parts being relatively movable in the axial direction of the tubular component.

42. A safety medical needle assembly as claimed in claim 41, wherein there is provided spring means to urge the first part of the movable component towards the second part, the first part having a cam surface engageable with the control member to turn the control member under the action of the spring means, once the control member has been turned through said predetermined angle.

43. A safety medical needle assembly as claimed in claim 42, wherein the cam surface of the first part engages the moving camming part of the control member, following movement of the control member through said predetermined angle.

44. A safety medical needle assembly as claimed in claim 30, wherein the tubular component has a plurality of similar parallel guides formed internally of the component in an angularly-spaced disposition.

45. A safety medical needle assembly as claimed in claim 44, wherein each guide provides said abutment surface behind which the control member engages to block movement of the movable component towards its shifted position.

46. A safety medical needle assembly as claimed in claim 30, wherein the medical needle has a hub at its rear end, the hub being removably mounted in the movable component and being configured to receive the forward part of a syringe to be connected thereto.

47. A safety medical needle assembly as claimed in claim 30 in combination with a syringe connected to a needle supported in the safety needle assembly.
